(19) United States
(12) Patent Application Publication
(10) Pub. No.: US 2005/0038319 A1
(43) Pub. Date: Feb. 17, 2005

(54) Gastrointestinal tool over guidewire

(76) Inventors: Benad Goldwasser, Tel Aviv (IL); Oz Cabiri, Macabim (IL); Yosef Gross, Morhav Mazor (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/747,648

(22) Filed: Dec. 30, 2003

Related U.S. Application Data
(63) Continuation-in-part of application No. 10/639,442, filed on Aug. 13, 2003.

(51) Int. Cl. A61B 1/00

(52) U.S. Cl. 600/104; 600/114; 600/109; 600/7; 604/93.01

(57) ABSTRACT

Gastrointestinal apparatus including a guidewire, and a gastrointestinal tool including a guiding member formed with a bore, the guidewire passing through the bore.

### CROSS-REFERENCE TO RELATED APPLICATIONS

This application is a continuation-in-part of U.S. patent application Ser. No. 10/639,442, filed Aug. 13, 2003, and assigned to the common assignee of the present invention.

### FIELD OF THE INVENTION

This invention relates generally to gastrointestinal tools, such as endoscopes and colonoscopes, and more particularly to gastrointestinal tools that slide over a guidewire, guiding catheter and the like, and which may be introduced into and through the colon, e.g., to the cecum, by any means.

### BACKGROUND OF THE INVENTION

Endoscopy has become an increasingly important tool in diagnosing and in treating ailments of the gastrointestinal tract, also referred to as the GI tract. Typical endoscopes are essentially formed by a somewhat flexible tube that is pushed through the GI tract, after being introduced in the body cavity starting from the rectum or starting from the esophagus. The endoscope has a steerable tip to facilitate navigation through the GI tract, and typically has to be sufficiently stiff so that it can be pushed further along the body cavity. The tip of the endoscope that is introduced in the GI tract can be outfitted with several devices, most notably an illumination device and a vision device, such as a vision integrated circuit, so that the operator of the endoscope can observe the interior of the GI tract and maneuver the endoscope in the proper position.

Once the endoscope is in position, other tools attached to the endoscope or inserted through the endoscope can be brought to the proper position in the GI tract. Various procedures can then be carried out, such as removing polyps, performing sutures, irrigation, suction, and removing other tissues. The various tools that are used together with the endoscope can be either inserted separately in the GI tract and placed in the proper position independently, or may travel in a working channel of the endoscope, so that once the endoscope is positioned at the desired location in the GI tract, the tools inserted in the endoscope will also easily reach that position.

Endoscopes or other smaller similar devices can also be used to explore other body cavities, for example airways, genitourinary tract, female reproductive organs, etc., or blood vessels. These probes must be small to fit in the smaller cavities, and care must be taken to avoid damage to the more fragile membranes lining these cavities.

Current state of the art endoscopes are very capable devices, and endoscopy has been very successful in diagnostic and therapeutic applications with the use of current endoscopes and the current arsenal of tools that can be inserted through the working channel of the endoscope, or can be attached to the outside of the endoscope. However, current endoscope technology has limitations and drawbacks. One of the greatest drawbacks of current endoscopes is that the working channel is small. The working channel is small relative to overall diameter of the endoscope, and is further limited by the space taken up by vision, irrigation, suction, light, and control cabling mechanisms that are part of the endoscope and are required to control the endoscope. Thus there is a very small area left for other tools to be introduced through the endoscope. Also, the additional channels may make passage of the endoscope through body cavities more difficult, as they contribute among other things to its diameter.

U.S. Pat. No. 6,517,477 to Wendlandt, assigned to Scimed Life Systems, Inc. (Maple Grove, Minn., US), describes a catheter introducer system for endoscopy that includes a steering section and a propulsion section located near the end of the flexible, tubular catheter that is introduced in a body cavity. The propulsion section is designed to pull the rest of the catheter inside the body cavity, so there is no need to push the catheter along from outside the body. Propulsion may be accomplished by relatively movable gripping pads that selectively apply suction to the tissue. The steering section is designed to point the end of the catheter that is introduced into the body cavity in the desired direction. The catheter may be made very flexible in bending, and a larger diameter catheter may be used.

### SUMMARY OF THE INVENTION

The present invention seeks to provide an improved gastrointestinal endoscope with tools that may be slid over a guidewire, as is described in detail hereinbelow.

It is noted that the term “guidewire” as used throughout the specification and claims, encompasses any instrument which may be introduced into a body lumen, such as but not limited to, a guiding catheter, monorail, wire, hollow or not hollow, with or without segments, and of any material property (e.g., flexibility).

There is thus provided in accordance with an embodiment of the present invention gastrointestinal apparatus including a guidewire, and a gastrointestinal tool including a guiding member formed with a bore, the guidewire passing through the bore.

The gastrointestinal apparatus may include one or more of the following features. For example, the guiding member may be positioned inwards or outwards of an outer contour of the gastrointestinal tool. The gastrointestinal tool may include a radioactive device (e.g., at least one of a radioactive tube, radioactive needle, radioactive seed, and radioactive capsule), an iontophoresis drug delivery device, an injection device for injection of a substance, a photodynamic therapy/diagnosis device, a hyperthermic therapy/diagnosis device, an ultrasonic therapy/diagnosis device, and/or an imaging device.

A propulsion device may be adapted to propel the gastrointestinal tool along the guidewire. An anchoring device (e.g., an inflatable balloon) may be adapted to anchor the guidewire at a position in the gastrointestinal tract. Additionally or alternatively, the gastrointestinal tool may be coupled to a pulley.

### BRIEF DESCRIPTION OF THE DRAWINGS

The present invention will be understood and appreciated more fully from the following detailed description taken in conjunction with the appended drawings in which:

FIG. 1 is a simplified illustration of a guidewire, in accordance with an embodiment of the present invention, introduced through the rectum into the large intestine all the way to the cecum;

FIG. 2 is a simplified illustration of an imaging device and sleeve slid over the guidewire of FIG. 1, in accordance with an embodiment of the present invention;

FIG. 3 is a simplified illustration of additional devices introduced over the guidewire of FIG. 1, such as but not limited to, a catheter with a lumen for passing therethrough wires (electrical or other) and another lumen for introducing therethrough fluids, or other gastrointestinal tools and the like, in accordance with an embodiment of the present invention;

FIG. 4 is a simplified illustration of a gastrointestinal tool slid over the guidewire of FIG. 1, in accordance with an embodiment of the present invention; and

FIG. 5 is a simplified illustration of a gastrointestinal tool with a pulley for movement over the guidewire of FIG. 1, in accordance with an embodiment of the present invention.

### DETAILED DESCRIPTION OF PREFERRED EMBODIMENTS

Reference is now made to FIGS. 1 and 2, which illustrate gastrointestinal apparatus 10, constructed and operative in accordance with an embodiment of the present invention.

Gastrointestinal apparatus 10 may comprise a gastrointestinal tool 12 formed with a bore 14 that slides over a guidewire 16. The guidewire 16 passes through bore 14. Examples of gastrointestinal tools 12 are described hereinbelow. Guidewire 16 may be constructed of any suitable flexible, medically safe material, such as a plastic or metal.

The following description follows for introducing guidewire 16 into the gastrointestinal tract, particularly the colon. However, it is emphasized that the invention is not limited to the GI tract and may be used for any other body lumen.

As seen in FIG. 1, guidewire 16 may be introduced through the rectum 18, past the sigmoid colon 20 and descending colon 22, where it may bend past the splenic flexure 24, pass through the transverse colon 26, bend past the hepatic flexure 28, pass into the ascending colon 30 and reach the cecum 32 by any method. For example, guidewire 16 may be pushed through the colon by hand as in typical colonoscopic procedures. Alternatively, guidewire 16 may be pulled through the colon, such as by a self-propelled mechanical device (or other devices, such as but not limited to, inflatable devices, electrical devices and the like). Gastrointestinal tool 12 may then be introduced into the colon by sliding over guidewire 16. Gastrointestinal tool 12 may be formed with a distal chamfer 34 to facilitate movement distally into the colon. Additionally, gastrointestinal tool 12 may be formed with a proximal chamfer 36 to facilitate extraction from the colon.

A pushing device 38, such as but not limited to, a sleeve or catheter, may be slid over guidewire 16 to distally push gastrointestinal tool 12 over guidewire 16. Guidewire 16 may comprise a distal stop 40 that prevents distal movement of gastrointestinal tool 12 therepast. Distal stop 40 may be inflatable, wherein inflation of distal stop 40 may be useful in fixing the position of guidewire 16 in the colon.

In accordance with an embodiment of the present invention, gastrointestinal tool 12 may comprise an imaging device. The imaging device may comprise, without limitation, a CCD camera, an illumination device, a vision device, an ultrasound sensor, and/or an x-ray emitter.

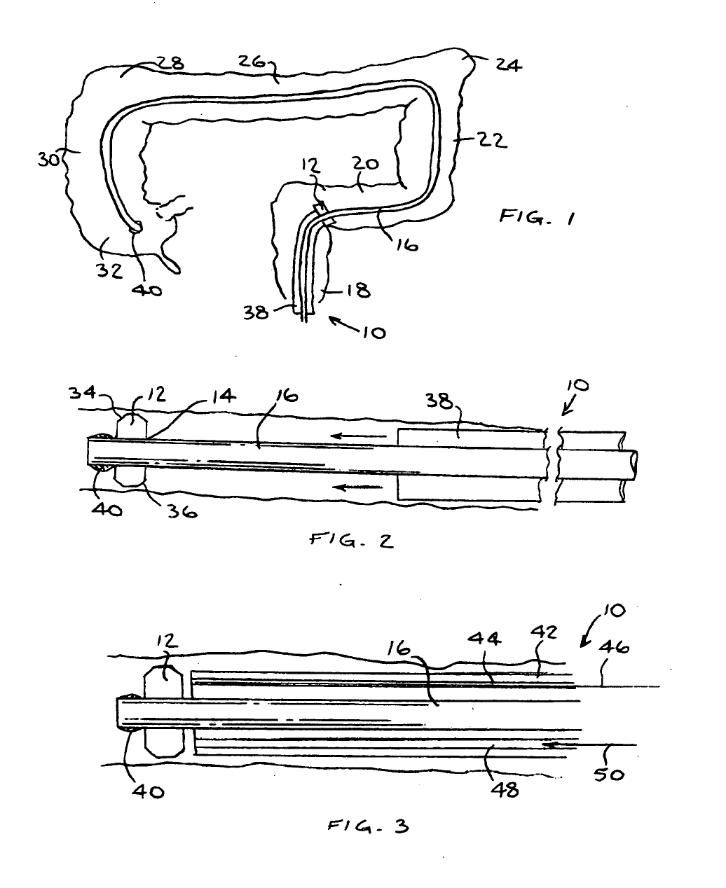

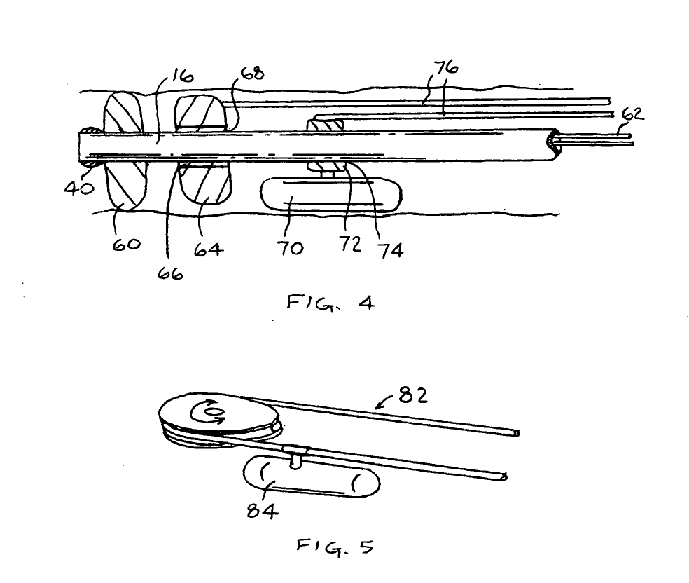

Reference is now made to FIG. 3. The gastrointestinal tool may further comprise a catheter 42 that slides over guidewire 16. Catheter 42 may comprise a lumen (also referred to as a channel) 44 for passing therethrough an instrument 46. Additionally or alternatively, catheter 42 may comprise a lumen (also referred to as a channel) 48 for passing therethrough a fluid 50. Catheter 42 may be rigid or flexible, and may have a one-piece or multiple-piece construction. Catheter 42 may comprise an inflatable, collapsible sleeve, as is described hereinbelow.

Catheter 42 may include any number of lumens for multiple applications. Catheter 42 may be constructed of any suitable medically safe material, such as a plastic or metal. Catheter 42 may have a generally fixed form, or may be made of a collapsible material (e.g., nylon), wherein after partial or complete introduction into the body lumen, catheter 42 may be inflated to functionally create an endoscope with single or multiple channels that run through its length.

In accordance with another embodiment of the present invention, instrument 46 or gastrointestinal tool 12 may comprise a cutting tool or a sampling device for performing a variety of medical procedures, such as but not limited to, removing polyps, performing sutures, irrigation, suction, and removing other tissues.

In accordance with an embodiment of the present invention, gastrointestinal tool 12 may comprise a magnetic device, such as for coupling with other GI devices. For example, the magnetic device may be used to magnetically attract magnetic boluses or other ingestible objects used for tracking or imaging the GI tract. Alternatively, gastrointestinal tool 12 may comprise any other suitable attachment or attraction device, such as but not limited to, adhesives or fasteners for attracting and affixing to such boluses or other ingestible objects.

In accordance with an embodiment of the present invention, substances may be introduced in the GI tract via gastrointestinal tool 12 and or guidewire 16. Such substances may include, without limitation, materials injected for image contrast or labeling, such as but not limited to, x-ray dyes, radioactivity-tagged materials or radiopharmaceuticals, magnetic resonance imaging (MRI) contrast agents, and others, or pharmaceuticals, relaxants, and other medicinal substances. As another example, a fluid (e.g., air) may be introduced in the GI tract via gastrointestinal tool 12 and or guidewire 16 for inflating the colon to enhance capturing images thereof.

The present invention thus provides a unique way of constructing a gastrointestinal apparatus by sliding gastrointestinal tool 12 over guidewire 16. Subsequently other tools may be slid over guidewire 16 to cooperate with gastrointestinal tool 12. As mentioned before, a collapsible sleeve (e.g., which may take the form of catheter 42 shown in FIG. 3) may be slid over guidewire 16 and inflated to functionally create an endoscope with single or multiple channels (e.g., 44 or 48) that run through its entire or partial length.

Reference is now made to FIG. 4, which illustrates various gastrointestinal tools slid over guidewire 16, in accordance with an embodiment of the present invention.

In the illustrated embodiment, an anchoring device 60 is provided to anchor guidewire 16 at any desired position in the GI tract. The anchoring device 60 may be a balloon inflatable by means of pressurized gas or liquid introduced through a lumen 62 in fluid communication with the balloon.

Different kinds of gastrointestinal tools may be slid over guidewire 16 as mentioned hereinabove. Two examples of possible configurations of such tools are shown schematically and simplistically in FIG. 4. In one configuration, a gastrointestinal tool 64 has a guiding member 66 positioned inwards of the outer contour of gastrointestinal tool 64. Guiding member 66 may be a sleeve or disc, for example, formed with a bore 68. Guidewire 16 passes through bore 68. In another configuration, a gastrointestinal tool 70 has a guiding member 72 positioned outside the outer contour of gastrointestinal tool 70. Guiding member 72 may be an eyelet or small tube attached to the body of the gastrointestinal tool 70. Guiding member 72 may be formed with a bore 74 through which guidewire 16 passes through. In such a case, the guidewire 16 acts as a “monorail” with the gastrointestinal tool 70 gliding along it like a cable car. In the second configuration, the gastrointestinal tool 70 may comprise a capsule or colonoscope, for example.

There are many kinds of gastrointestinal tools that may be configured in either of the two configurations just described. (The tools are shown schematically and simplistically in FIG. 4, and it is noted that the shape and size of the tools may not be as exactly shown in the figure.) Some non-limiting examples of such gastrointestinal tools include a radioactive device (e.g., a radioactive tube, radioactive needle, radioactive seed, and/or radioactive capsule or combination thereof), or an iontophoresis drug delivery device. The iontophoresis drug delivery device may comprise at least two electrodes in intimate electrical contact with some portion of the GI tract, wherein one electrode is the active or donor electrode, from which an ionic substance, agent, medicament, drug precursor or drug is delivered into the body via the wall of the GI tract by iontophoresis. The other electrode, called the counter or return electrode, serves to close the electrical circuit through the body. The circuit is completed by connection of the electrodes to a source of electrical energy, e.g., a battery. One or both of the anode and the cathode may be used to deliver drugs of appropriate charge into the body.

Other non-limiting examples of such gastrointestinal tools include an imaging device (e.g., miniature CCD or x-ray imaging device), a miniature injection device for injection of a substance (e.g., an endoscopically-actuated injection needle and/or syringe), a photodynamic therapy/diagnosis device (e.g., an LED-based light infusion device), a hyperthermic therapy/diagnosis device (e.g., miniature high frequency ultrashort wave electrodes), or an ultrasonic therapy/diagnosis device (e.g., miniature ultrasound transducer). The imaging or viewing device may be used to position the gastrointestinal tool at any desired position along the guidewire 16.

In the case of diagnostic applications, energy, such as ultrasonic or x-ray energy, is applied to at least a portion of the GI tract to image the target tissue. A visible image of an internal region of the GI tract is then obtained, such that the presence or absence of diseased tissue can be ascertained. With respect to ultrasound, ultrasonic imaging techniques, including second harmonic imaging, and gated imaging, are well known in the art. Ultrasound can be used for both diagnostic and therapeutic purposes. In diagnostic ultrasound, ultrasound waves or a train of pulses of ultrasound may be applied with a transducer. The ultrasound is generally pulsed rather than continuous, although it may be continuous, if desired. Diagnostic ultrasound generally involves the application of a pulse of echoes, after which, during a listening period, the ultrasound transducer receives reflected signals. Harmonics, ultraharmonics or subharmonics may be used. Energy levels for therapeutic ultrasound may be used to perform hyperthermia.

Photodynamic therapy/diagnosis utilizes the administration of a light-sensitive drug, which is then exposed to a visible light beam, which, in the case of therapy, may destroy cancerous cells. It is known that certain dyes are accreted by lesions, such as tumors, which are in turn activated by a specific frequency of light. The photodynamic therapy/diagnosis device may employ laser-induced fluorescence to detect sites of cancer which have accreted the light-activated dye. Fluorescent and other chromagens, or dyes, such as porphyrins sensitive to visible light, have been used to detect and even treat lesions by directing the suitable light to the tumor or lesion. Other synonymous names for photodynamic therapy/diagnosis device include photoradiation and phototherapy.

Another gastrointestinal tool may include a dye applicator, which may be used to coat, paint, spray or otherwise apply a color, pigment, dye or other highlighting substance on the inner walls of the GI tract. In this manner, polyps or other growths or anomalies may be highlighted so that they are more easily discerned and imaged by the colonoscope. This may also aid in pattern recognition by the colonoscope. It is also noted that certain polyps or other growths may react differently to the addition of the dye, such as in terms of color change, absorption, etc., and the reaction of the growth may be correlated to its probability of turning into a cancerous growth.

A propulsion device 76 may be provided to propel (e.g., push or pull) the gastrointestinal tool along guidewire 16. For example, the propulsion device 76 may include a push and/or pull wire attached to the tool. In another non-limiting example, the propulsion device 76 may include a jet-propulsion device that moves the tool distally over the guidewire 16 by means of a jet of water or air expelled proximally from the jet-propulsion device. As another example, the propulsion device 76 may be a motor with gearing which may not need any external connections, which moves the gastrointestinal tool along guidewire 16.

Reference is now made to FIG. 5, which illustrates a gastrointestinal tool 80 with another kind of propulsion device, in accordance with an embodiment of the present invention. In this embodiment, the propulsion device includes a pulley 82 for movement over guidewire 16. A gastrointestinal tool 84 may be coupled to pulley 82 and moved distally and/or proximally along guidewire 16. The pulley 82 may be connected to the end of guidewire 16 or any other portion thereof, or to any other device that moves along or with the catheter (the catheter being guidewire 16).

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

1. Gastrointestinal apparatus comprising:
a guidewire; and
a gastrointestinal tool comprising a guiding member formed with a bore, said guidewire passing through said bore.

2. The gastrointestinal apparatus according to claim 1, wherein said guiding member is positioned inwards of an outer contour of said gastrointestinal tool.

3. The gastrointestinal apparatus according to claim 1, wherein said guiding member is positioned outside an outer contour of said gastrointestinal tool.

4. The gastrointestinal apparatus according to claim 1, wherein said gastrointestinal tool comprises a radioactive device comprising at least one of a radioactive tube, radioactive needle, radioactive seed, and radioactive capsule.

5. The gastrointestinal apparatus according to claim 1, wherein said gastrointestinal tool comprises an iontophoresis drug delivery device.

6. The gastrointestinal apparatus according to claim 1, wherein said gastrointestinal tool comprises an injection device for injection of a substance.

7. The gastrointestinal apparatus according to claim 1, wherein said gastrointestinal tool comprises a photodynamic therapy/diagnosis device.

8. The gastrointestinal apparatus according to claim 1, wherein said gastrointestinal tool comprises a hyperthermic therapy/diagnosis device.

9. The gastrointestinal apparatus according to claim 1, wherein said gastrointestinal tool comprises an ultrasonic therapy/diagnosis device.

10. The gastrointestinal apparatus according to claim 1, further comprising a propulsion device adapted to propel said gastrointestinal tool along said guidewire.

11. The gastrointestinal apparatus according to claim 1, further comprising an anchoring device adapted to anchor said guidewire at a position in a gastrointestinal tract.

12. The gastrointestinal apparatus according to claim 11, wherein said anchoring device comprises an inflatable balloon.

13. The gastrointestinal apparatus according to claim 1, wherein said gastrointestinal tool is coupled to a pulley.

14. The gastrointestinal apparatus according to claim 1, wherein said gastrointestinal tool comprises an imaging device.
